(19) United States
(12) Patent Application Publication
(10) Pub. No.: US 2025/0283133 A1
(43) Pub. Date: Sep. 11, 2025

(54) METHOD TO REDUCE DOUBLE STRANDED RNA BY-PRODUCT FORMATION

(71) Applicant: eTheRNA immunotherapies NV, Niel (BE)

(72) Inventors: Phillip CHALLIS, Niel (BE); Lubos BAUER, Niel (BE)

(21) Appl. No.: 18/561,864

(22) PCT Filed: May 25, 2022
(86) PCT No.: PCT/EP2022/064233

§ 371 Date: Nov. 17, 2023

(30) Foreign Application Priority Data
May 26, 2021 (EP) 21175889.1

(51) Int. Cl. C12P 19/34

(52) U.S. Cl. CPC C12P 19/34

(57) ABSTRACT

The present invention relates to the field of nucleic acid production, in particular in vitro RNA transcription. More specifically, the present invention relates to a method to reduce formation of double stranded RNA during in vitro transcription, more in particular by the use of particular amounts of Mg during the RNA transcription process. The invention further relates to an in vitro transcribed RNA composition obtainable by the method according to the invention.

### CROSS-REFERENCES TO RELATED APPLICATIONS

This application is a national-stage entry under 35 U.S.C. § 371 of International Application PCT/EP2022/064233, filed May 25, 2022, which International Application claims the benefit of priority to European Patent Application No. 21175889, filed May 26, 2021.

### TECHNICAL FIELD

The present disclosure relates to the field of nucleic acid production, in particular in vitro RNA transcription. More specifically, the present disclosure relates to a method to reduce formation of double stranded RNA during in vitro transcription, more in particular by the use of particular amounts of Mg during the RNA transcription process. The disclosure further relates to an in vitro transcribed RNA composition obtainable by the method according to the disclosure.

### BACKGROUND

One prominent use of in vitro transcription (IVT) has been to generate mRNAs for biopharmaceutical and therapeutic applications. The simplicity of the approach, that is, synthesizing an mRNA in vitro that resembles an endogenous mRNA and encodes a protein of interest, followed by its delivery and expression in vitro or in vivo, makes it appealing and has broad applicability.

The technology used for the (large scale) synthesis of IVT RNAs is robust and well established. One important drawback is the presence or generation of certain by-products of the in vitro synthesis process, including double-stranded RNA (dsRNA), that trigger cellular immune responses. The application of IVT RNA for use as a therapeutic requires large amounts of functional RNA with low immunogenicity. Therefore, when synthesizing mRNAs for in vivo applications that seek to minimize cellular immune responses, it is critical to either eliminate these dsRNA contaminants from the mRNA preparations or reduce dsRNA formation.

Several methods have been described that rely on the removal of the by-products after completion of the IVT reaction, with analytical purification such as chromatography-based purification methods being the most predominant approaches. Although efficient, this approach results in an additional step in the mRNA synthesis workflow, involves specialized instrumentation, is often not compatible with upscaling of the reaction, may reduce RNA yield and impedes the cost effectiveness of the approach. An alternative approach to post-synthesis purification is to prevent formation of the dsRNA by-products in the IVT reaction by altering the IVT reaction conditions. Lowering the magnesium levels in the IVT reaction has been suggested to reduce the formation of dsRNA by-products (formed by synthesis of antisense RNA) for a few specific templates (Mu et al. 2018); however, lowering the magnesium concentration in the reaction also affects the total yield of RNA, which is undesirable for applications where large quantities of mRNA are desired. Moreover, the dsRNA detection method used by Mu et al., 2018 (i.e. native gel electrophoresis) is biased towards large fragments of dsRNA, whereas short dsRNA fragments may remain undetected using this method. Accordingly, the results of Mu et al., 2018 do not allow to draw any conclusions on the total amount of dsRNA in a reaction.

The inventors of the present disclosure have unexpectedly found that dsRNA by-product formation can be reduced during in vitro transcription (IVT) with—in contrast to Mu et al 2018—an elevated concentration of magnesium in the reaction. In particular, the present disclosure describes a method for reducing double stranded RNA (dsRNA) formation during in vitro transcription in the presence of at least about 35 mM of magnesium—compared to conventional concentrations of approximately 19 mM of magnesium. The main advantage of this method to produce IVT RNAs is a reduction of 50-70% of total dsRNA while the yield and integrity of the produced RNA is not compromised. In addition, the described method is compatible with the manufacturing process for different kind of mRNAs including uncapped RNA, CAP-0 and CAP-1 capped RNA, nucleoside modified RNA (e.g. N1-methyl pseudouridine modified), RNA with and without a polyA tail.

The method best suited for the removal/prevention of the dsRNA contaminants will depend on the final application and the scale of RNA yield desired. For applications where upscaling is a prerequisite, a post-synthesis purification step can impede the final outcome. Therefore, this disclosure provides a solution to prevent or at least reduce the formation of the dsRNA by-products during the synthesis process.

### SUMMARY

In a first aspect, the present disclosure provides a method for reducing double stranded RNA (dsRNA) formation during an in vitro transcription (IVT) reaction comprising performing said IVT reaction in the presence of at least about 35 mM of magnesium.

In a further embodiment, said IVT reaction is performed in the presence of pyrophosphatase.

In another embodiment, said IVT transcription reaction is terminated by addition of a metal chelator, such as EDTA.

In a specific embodiment of the present disclosure, the concentration of magnesium is about and between 35 mM to about 150 mM, preferably about and between 40 mM and about 100 mM, more preferably about and between 45 mM and about 75 mM, most preferably about 55 mM.

In another specific embodiment of the method of the present disclosure, the concentration of pyrophosphatases is about and between 0.01 U/ml to about 40 U/ml, preferably about and between 0.1 U/ml and about 20 U/ml, more preferably about and between 1 U/ml and about 10 U/ml, most preferably about 5 U/ml.

In yet another specific embodiment, the concentration of the concentration of said metal chelator is about and between 10 and about 50 mM, preferably about and between 20 and about 30 mM, more preferably about 24 mM.

In yet a further embodiment, magnesium can be in any salt form comprising magnesium chloride (MgCl₂), magnesium acetate (MgOAc₂).

In a particular embodiment, RNA in said in vitro transcription reaction may further comprise one or more of the following: a 5′ CAP, modified nucleoside(s), and/or a poly(A) tail.

In a further aspect, the present disclosure provides the use of at least about 35 mM magnesium in an IVT reaction to reduce the formation of double stranded RNA (dsRNA) during said IVT reaction.

### BRIEF DESCRIPTION OF THE DRAWINGS

With specific reference now to the figures, it is stressed that the particulars shown are by way of example and for purposes of illustrative discussion of the different embodiments of the present disclosure only. They are presented in the cause of providing what is believed to be the most useful and readily description of the principles and conceptual aspects of the disclosure. In this regard no attempt is made to show structural details of the disclosure in more detail than is necessary for a fundamental understanding of the disclosure. The description taken with the drawings making apparent to those skilled in the art how the several forms of the disclosure may be embodied in practice.

FIG. 1: Amount of dsRNA detected after an in vitro transcription at a concentration of 24 vs 55 mM magnesium for RNA with no cap (panel A) and CleanCap (cap-1) (panel B).

Using 55 mM Mg reduced the amount of dsRNA with an average of 62% (average panel A and B) compared with reactions using 24 mM Mg. Striped bars represent the 24 mM Mg concentration while solid filled bars represent 55 mM Mg concentration. The same samples are aligned next to each other but treated with a different amount of Mg (respectively 24 mM vs. 55 mM Mg). All five tested mRNAs have a different open reading frame and share a uniform polyA tail in a length of 120 nucleotides.

FIG. 2: Developed band intensities of an immunoblot utilizing the anti-dsRNA J2 antibody of samples after in vitro transcription reaction treated with 24 mM and 55 mM Mg.

The arrow indicate the same samples but treated with a different amount of Mg (respectively 24 mM vs. 55 mM Mg).

FIG. 3: In vitro transcription reaction yield (μg) at a concentration of 24 vs 55 mM magnesium for RNA with no cap (panel A) and CleanCap (cap1) (panel B).

Striped bars represent the 24 mM Mg concentration while solid filled bars represent 55 Mg concentration. The same samples are aligned next to each other but treated with a different amount of Mg (respectively 24 mM vs. 55 mM Mg).

### DETAILED DESCRIPTION

The present disclosure will now be further described. In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed embodiments. It is to be understood that the value to which the modifier “about” or “approximately” refers is itself also specifically, and preferably, disclosed.

The present disclosure relates to a method to reduce formation of dsRNA during an IVT reaction. The disclosure further relates to a purified in vitro transcribed RNA composition obtainable by the method according to the disclosure. The inventors of the present disclosure have unexpectedly found that dsRNA by-product formation can be reduced during IVT with an optimum concentration of magnesium in the reaction. In particular, the present disclosure describes a method for reducing dsRNA formation during in vitro transcription in the presence of at least about 35 mM of magnesium-compared to conventional concentrations of approximately 19 mM of magnesium. The main advantage of this method to produce IVT RNAs is a reduction of 50-70% of dsRNA while the yield and integrity of the produced RNA is not compromised. In addition, the described method is compatible with the manufacturing process for different kind of mRNAs including uncapped RNA, CAP-0 and CAP-1 (Cleancap) RNA, nucleoside modified RNA (e.g. N1-methyl pseudoruidine), RNA, or RNA with and without a polyA tail.

In a first aspect, the present disclosure provides a method for reducing double stranded RNA (dsRNA) formation during an in vitro transcription (IVT) reaction comprising performing said IVT reaction in the presence of at least about 35 mM of magnesium.

In the context of the present disclosure, the terms ‘reducing’ or alternatively ‘to reduce’ are meant to be to ‘lessen’, to ‘decrease’, to ‘minimize’, or to ‘diminish’ the formation of dsRNA. Accordingly, where a sample would under normal circumstance contain a particular amount of dsRNA after in vitro transcription, the term ‘reducing’ means that said amount of dsRNA is lower when subjecting said sample to the method of the present disclosure. In particular, the amount of dsRNA is preferably reduced by at least 10%, such as at least 20%, at least 30%, at least 40%, at least 50%, at least 60%, at least 70%, at least 80%, at least 90%, when compared to normal circumstances.

In the context of the present disclosure, the term ‘the formation’ is meant to be ‘the emergence’, ‘the development’, ‘the origination’, or ‘the generation’ of dsRNA in said in vitro transcription reaction. Specifically, molecules obtained after in vitro transcription typically comprise dsRNA, while we have identified that the presence of elevated magnesium in the reaction results in a reduced formation of such dsRNA.

In the context of the present disclosure, the term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. In particular, the term refers to double stranded RNA, but may also refer to single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

According to the present disclosure, the term “RNA” includes and preferably relates to “mRNA” which means “messenger RNA” and relates to a “transcript” which may be produced using DNA as template and encodes a peptide or protein. mRNA typically comprises a 5′ untranslated region (5′-UTR), a protein or peptide coding region and a 3′ untranslated region (3′-UTR). mRNA has a limited halftime in cells and in vitro.

The term ‘modified mRNA molecules’ means mRNA molecules that contain one or more modified nucleosides (termed “modified nucleic acids”), which have useful properties such as the lack of a substantial induction of the innate immune response of a cell into which the mRNA is introduced. These modified nucleic acids enhance the efficiency of protein production, intracellular retention of nucleic acids, and viability of contacted cells, as well as possess reduced immunogenicity. An exemplary suitable modified nucleoside may for example by N1-methyl pseudouridine.

For the sake of clarity, a mRNA encompasses any coding RNA molecule, which may be translated by an eukaryotic host into a protein.

Preferably, mRNA is produced by in vitro transcription using a DNA template. In one embodiment of the disclosure, the RNA is obtained by in vitro transcription. The in vitro transcription methodology is known to the skilled person and may comprise a purified linear DNA template containing a promoter, ribonucleotide triphosphates, a buffer system that includes dithiothreitol (DTT) and magnesium ions, spermidine and an appropriate RNA polymerase such as T7 RNA polymerase. The exact conditions used in the transcription reaction depend on the amount of RNA needed for a specific application. There is a variety of in vitro transcription kits commercially available.

In the context of the present disclosure, the term “double stranded RNA” or “dsRNA” is meant to be any RNA molecule with sufficient internal homology to form significant secondary structures such as hairpins due to hybridization of internal complementary sequences with one another via Watson-Crick base pairing of nucleotide bases within the complementary sequences. Significant secondary structures generally involve stretches of homology greater than approximately nine bases, but the exact length depends to some extent on context and on whether such secondary structures impart any biological function to the molecule. In particular, molecules obtained after in vitro transcription typically comprise dsRNA with two separate complementary strands and may vary in size for example from 20 nucleotides to 200 nucleotides or even more than 500 nucleotides.

In the context of the present disclosure, dsRNA is formed as a byproduct identified in IVT reactions which can arise from T7 RNA-dependent RNA polymerase activity. In particular, three main types of byproduct in the IVT reaction may result in formation of dsRNA molecules. The first is formed by 3′-extension of the run-off products annealing to complementary sequences in the body of the run-off transcript either in cis (by folding back on the same RNA molecule) or trans (annealing to a second RNA molecule) to form extended duplexes. The second type of dsRNA molecules is formed by hybridization of an antisense RNA molecule to the run-off transcript. The antisense RNA molecules have been reported to be formed in a promoter- and run-off transcript-independent manner. Alternatively, a promoter-independent transcription of full-length anti-sense RNA has been also reported as a novel mechanism of dsRNA generation in T7 RNAPol-driven IVT reaction. A third form of dsRNA results from random pairing of abortive transcripts, either in cis (i.e. within the same molecule) or in trans (between two different molecules). According to the disclosure, dsRNA encompasses any kind of the described RNA byproducts in an IVT reaction.

Methods for detecting dsRNA rely essentially on immunological approaches such as immunofluorescence, ELISA, immunoblot as well as antibody-independent methods such as nucleic acid fluorescent in situ hybridization (FISH) or cellulose-based dsRNA isolation have also been used for dsRNA detection. As used herein and as described in the examples, immunological methods such as anti dsRNA J2 antibody immunoblotting, use antibodies as structural probes that specifically recognize the A-helix structure adopted by dsRNA. Commercially available J2 anti-dsRNA IgG2a (and to a lesser extent the IgG2a K1 and IgM K2 mAb or 9D5 mAb) have become the golden standards in dsRNA detection. Furthermore, intact mass spectrometry can be used to quantify the abundance and lengths of different 3′-end-extended dsRNA species.

As used herein and unless otherwise specified, the term “magnesium” or “Mg” is to be understood as a chemical element essential to the basic nucleic acid chemistry of all cells of all known living organisms. More than 300 enzymes require magnesium ions for their catalytic action, including enzymes using or synthesizing ATP and those that use other nucleotides to synthesize DNA and/or RNA. According to the disclosure, Mg²⁺ ions are provided by any of the described magnesium forms and are needed to catalyze the reactions driven by for example RNA polymerases such as T3, T7, SP6, the pyrophosphatase and the DNAse I. Accordingly, this component needs to be provided throughout the whole reaction and has a specific function for the enzymes and hence influences IVT yield. It was particularly found that adding elevated concentration of Mg to the IVT reaction preferably about and between 35 mM to about 150 mM, preferably about and between 40 mM and about 100 mM, more preferably about and between 45 mM and about 75 mM, most preferably about 55 mM resulted in a reduced formation of dsRNA preferably reduced by at least 10%, such as at least 20%, at least 30%, at least 40%, at least 50%, at least 60%, at least 70%, at least 80%, at least 90% when compared to normal circumstances.

In particular, the ratio of magnesium to NTPs is a critical parameter influencing efficient IVT through the catalytic activity of T7 polymerase. For example, a combination of 10 mM of each NTP with 75 mM of magnesium anion produced optimal IVT RNA yield, where 5 mM of each NTP is half as good and 20 mM of each NTP being too much.

In a particular embodiment, the yield after the IVT reaction is not compromised or preferably increased by at least 5%, at least 10%, at least 15%, at least 20%, at least 25%, at least 30%, at least 35%, at least 40%, at least 45%, at least 50%, at least 60%, at least 70%, at least 80%, at least 90%, at least 100%, at least 120%, at least 140%, at least 160%, at least 180%, at least 200%, or even more when compared to normal circumstances. In particular, higher amounts of Magnesium were found to significantly increase the yield of RNA from the IVT reaction.

In a specific embodiment, the integrity of RNA after the IVT reaction is not compromised or preferably increased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% or even higher, when compared to normal circumstances. The integrity of the RNA can be measured by any suitable means such as by capillary electrophoresis peak profiles, which may be obtained on a bioanalyzer. Specifically, no significant degradation was observed in the experiments performed herein and peak profiles were nearly identical for conditions using 24 mM vs 55 mM magnesium.

In a specific embodiment of the present disclosure, the concentration of magnesium is about and between 35 mM to about 150 mM, preferably about and between 40 mM and about 100 mM, more preferably about and between 45 mM and about 75 mM, most preferably about 55 mM.

In some embodiments, said concentration of magnesium may be at least about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 mM. In a particular embodiment, the concentration of magnesium present is preferably about 35 mM, about 45 mM or about 55 mM.

In some embodiments, the presence of about 35 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 40 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 45 mM magnesium in the IVT reaction reduces the formation of dsRNA for at least 10%, at least 20%, at least 30%, at least 40%, at least 50%, at least 60%, at least 70%, at least 80%, at least 90%, at least 100% when compared to normal circumstances.

In some embodiments, the presence of about 50 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 55 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 60 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 65 mM magnesium in the IVT reaction reduces the formation of dsRNA for at least 10%, at least 20%, at least 30%, at least 40%, at least 50%, at least 60%, at least 70%, at least 80%, at least 90 when compared to normal circumstances.

In some embodiments, the presence of about 70 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 75 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 80 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 85 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 90 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 95 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In some embodiments, the presence of about 100 mM magnesium in the IVT reaction reduces the formation of dsRNA for at least 10%, at least 20%, at least 30%, at least 40%, at least 50%, at least 60%, at least 70%, at least 80%, at least 90% when compared to normal circumstances.

In yet a further embodiment, magnesium is in a form selected from the group comprising magnesium chloride (MgCl₂), magnesium acetate (MgOAc₂).

In a particular embodiment, magnesium is in a form selected from the group comprising magnesium chloride, magnesium acetate, magnesium sulfate, magnesium hydroxide, magnesium oxide, magnesium gluconate, magnesium malate, magnesium orotate, magnesium glycinate, magnesium ascorbate, magnesium citrate, magnesium borate, magnesium salicylate, magnesium bromide, magnesium stearate, magnesium carbonate, or any combination thereof.

In particular, during in vitro transcription (IVT) reaction magnesium is in the form of magnesium chloride.

In a further embodiment, said IVT reaction is performed in the presence of pyrophosphatase.

In another specific embodiment of the method of the present disclosure, concentration of pyrophosphatases is about and between 0.01 U/ml to about 40 U/ml, preferably about and between 0.1 U/ml and about 20 U/ml, more preferably about and between 1 U/ml and about 10 U/ml, most preferably about 5 U/ml.

In some embodiments, said concentration of pyrophosphatase may be at least about 0.01, U/ml. In a particular embodiment, the concentration of pyrophosphatase present is preferably about 5 U/ml.

As used herein, the term “pyrophosphatase”, also known as diphosphatase, is to be understood as acid anhydride hydrolases that act upon diphosphate bonds. The term preferably relates to inorganic pyrophosphatase which catalyzes the hydrolysis of inorganic pyrophosphate to form orthophosphate. Inorganic pyrophosphate is released when a nucleoside triphosphate is incorporated/polymerized into the growing chain. Pyrophosphate is an inhibitor of RNA polymerization and therefore, removal leads to an increase in RNA yield in IVT. Mg ions are necessary for catalytic activity of crystalline pyrophosphatase.

In a further aspect, pyrophosphatase may also be selected from the list comprising tobacco acid pyrophosphatase, which catalyses the hydrolysis of a phosphoric ester, various organic pyrophosphatases, which act upon organic molecules with the pyrophosphate group (but excluding triphosphatases that act on the final bond), thiamine pyrophosphatase.

In a specific embodiment of the present disclosure, said IVT transcription reaction is terminated by addition of a metal chelator such as selected from the list comprising: BAPTA (1,2-Bis (2-aminophenoxy) ethane-N,N,N′,N′-tetraacetic acid), DFOA (Deferoxamine Mesylate), Dimethoxynitrophenamine (1-(2-Nitro-4,5-dimethoxyphenyl)-1,2-diaminoethane-N,N,N′,N′-tetraacetic Acid), EDTA (ethylenediaminetetraacetic acid), EGTA (ethylene glycol-bis (β-aminoethyl ether)-N,N,N′,N′-tetraacetic acid), CDTA (1,2-cyclohexylenedinitrilo)tetraacetic acid), DPTA (diethylenetriaminepentaacetic acid), PIH (pyridoxal isonicotinoyl hydrazone), TPEN (N′-Tetrakis (2-pyridylmethyl)ethylenediamine).

In a specific embodiment, said IVT transcription reaction is terminated by addition of a metal chelator, such as EDTA.

As used herein, the term “EDTA” is to be understood as an aminopolycarboxylic acid acting as a scavenger for metal ions. This results in deactivation of metal-dependent enzymes, either as an assay for their reactivity or to suppress damage to DNA, proteins, and polysaccharides. In addition to metal ion chelation, EDTA also acts as a selective inhibitor against dNTP hydrolyzing enzymes such as Taq polymerase, dUTPase, MutT, etc.

In particular, as used in the present disclosure, EDTA chelates divalent cations such as magnesium and is needed to protect RNA from being degraded during enzyme inactivation. Nuclease activity and in particular RNA nuclease is highly dependent on the concentrations of divalent cation magnesium. In particular, it is known that one molecule of a metal chelator such as EDTA is capable of chelating one metal ion. The addition of metal chelators thus potentially has two benefits. On the one hand, it will stop enzymatic reactions that require the presence of metal ions as a cofactor, and secondly it will chelate metal ions thereby preventing the formation of the aggregate.

In yet another specific embodiment, the concentration of said metal chelator is about and between 10 and about 50 mM, preferably about and between 20 and about 30 mM, more preferably about 24 mM.

In a particular embodiment, RNA in said in vitro transcription reaction may be capped and uncapped RNA, modified and unmodified RNA, or RNA with and without poly (A) tail, or any combination thereof. A mature mRNA ready for efficient translation by the ribosome contains two major modifications: a 5′ cap structure and a poly (A) tail. Moreover, the IVT reaction using high amounts of Magnesium is not compromised towards short or long RNA molecules, i.e. it works equally well on small and long templates.

According to the disclosure, an RNA molecule, such as a messenger RNA (or mRNA), comprises the following types: uncapped unmodified RNA without poly (A) tail, uncapped unmodified RNA with poly (A) tail, uncapped modified RNA without poly (A) tail, uncapped modified RNA with poly (A) tail, capped unmodified RNA without poly (A) tail, capped unmodified RNA with poly (A) tail, capped modified RNA without poly (A) tail, capped modified RNA with poly (A) tail.

In the context of the present disclosure, the term “capped RNA” is to be understood as an RNA molecule of which the 5′ end is linked to a guanosine or a modified guanosine, preferably a 7-methylguanosine (N7-methyl guanosine or m7G), connected to a 5′ to 5′ triphosphate linkage or analogue. “Capping” of the RNA structure plays a crucial role in a variety of cellular processes which include translation initiation, splicing, intracellular transport and turnover. In vitro synthesis of capped mRNAs is performed by bacteriophage RNA polymerase (T7, SP6 or T3)-mediated in vitro transcription that co-transcriptionally incorporate cap analogues at the 5′-end of the transcripts. Alternatively, post-transcriptional enzymatic capping may also be used to add a 5′CAP to the IVT produced RNA molecules.

As used herein, “cap analogues” are caps which are biologically equivalent to a 7-methylguanosine (m7G), and comprise traditional analogues such as G (5′)ppp(5′) G, m7G (5′)ppp(5′) G or m2,2,7G (5′)ppp(5′) G, but also Anti-Reverse Cap Analog (ARCA) 3′-O-Me-m7G (5′)ppp(5′) G, Unmethylated Cap Analog G (5′)ppp(5′) G, Methylated Cap Analog for A+1 sites m7G (5′)ppp(5′) A; Unmethylated Cap Analog for A+1 sites G (5′)ppp(5′) A. Anti-Reverse Cap Analog (ARCA) is a modified cap analogue in which the 3′ OH group (closer to m7G) is replaced with —OCH3 that forces ARCA incorporation in the correct orientation and subsequently results in a translatable mRNA population.

In some preferred embodiments, the mRNA used in the methods of the present disclosure has a 5′ cap structure with a so-called CAP-1 structure (CleanCap), meaning that the 2′ hydroxyl of the ribose in the penultimate nucleotide with respect to the cap nucleotide is methylated, such as illustrated below:

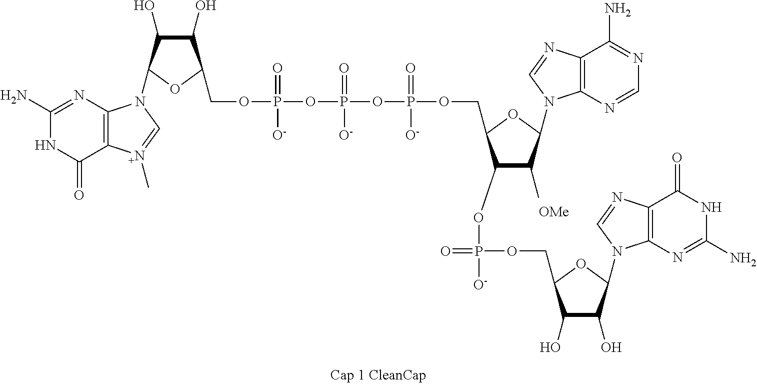

In the context of the present disclosure, the term “uncapped RNA” is to be understood as any RNA molecule that does not comprise a cap as defined in the definition “capped RNA”. Thus, in a particular embodiment, “uncapped mRNA” may refer to an mRNA of which the 5′ end is not linked to a 7-methylguanosine, through a 5′ to 5′ triphosphate linkage, or an analogue as previously defined.

In the context of the present disclosure, the term “modified RNA” is to be understood as an RNA molecule which contains at least one modified nucleotide, nucleoside or base, such as a modified purine or a modified pyrimidine. A modified nucleoside or base can be any nucleoside or base that is not A, U, C or G (respectively Adenosine, Uridine, Cytidine or Guanosine for nucleosides; and Adenine, Uracil, Cytosine or Guanine when referring solely to the sugar moiety).

In the context of the present disclosure, the term “unmodified RNA” is to be understood as any RNA molecule that does not comprise a modification as defined in the definition “modified RNA”. As used herein, the term “poly (A) tail” is to be understood as a moiety comprising multiple adenosine monophosphates and is well known in the art. A poly (A) tail is generally produced during a step called polyadenylation that is one of the post-translation modifications which generally occur during the production of mature messenger RNAs; such poly (A) tail contribute to the stability and the half-life of said mRNAs, and can be of variable length. In particular, a poly (A) tail may be equal or longer than 10 adenosine nucleotides, which includes equal or longer than 20 adenosine nucleotides, which includes equal or longer than 100 adenosine nucleotides, and for example about 120 adenosine nucleotides.

In the context of the present disclosure, the term “without poly (A) tail” is to be understood as any

RNA molecule that does not comprise a poly (A) tail as describe in the definition “poly (A) tail”. In the sense of the disclosure, the terms “modified and unmodified” are considered distinctly from “capped and uncapped”, as the latter specifically relates to the base at the 5′-end of a RNA molecule, and also distinctly from “with poly (A) tail and without poly (A) tail”.

In a further aspect, the present disclosure provides the use of at least about 35 mM magnesium in an IVT reaction to reduce the formation of double stranded RNA (dsRNA) during said IVT reaction.

### Examples

### Material and Methods

Crude IVT reactions or purified IVT RNA samples were spotted onto positively charged nylon membranes (Zeta-Probe blotting membrane, Bio-Rad). The membranes were blocked in 5% (w/v) non-fat dried milk in TBS-T buffer (20 mM Tris, pH 7.4, 150 mM NaCl, 0.05% (v/v) Tween-20). For the detection of dsRNA, the membranes were incubated with J2 anti-dsRNA antibody (1:5000; Scicons) at 4° C. overnight. The blots were probed with Goat pAb to Ms IgG2a, HRP (Abcam), dsRNA standard dilution (1000 bp) was used to generate a linear standard curve.

### Results

dsRNA Reduction

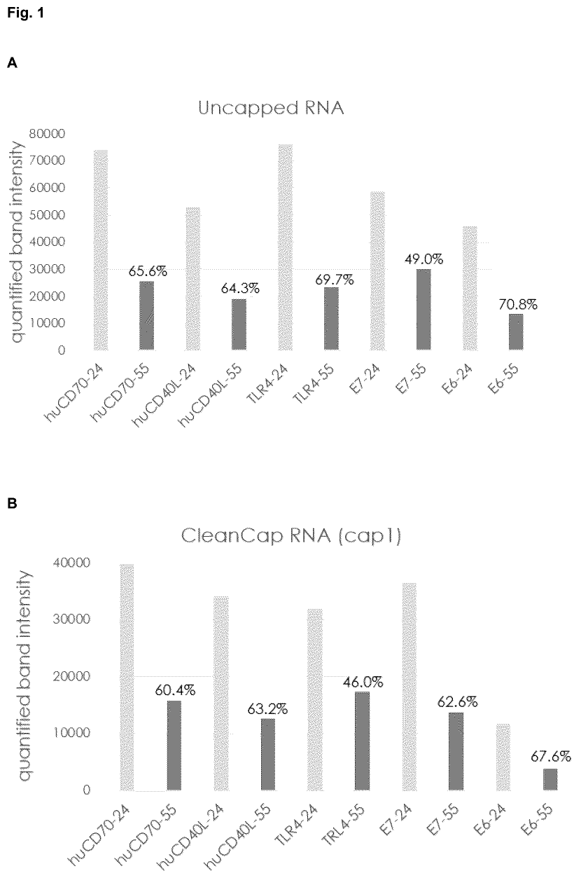

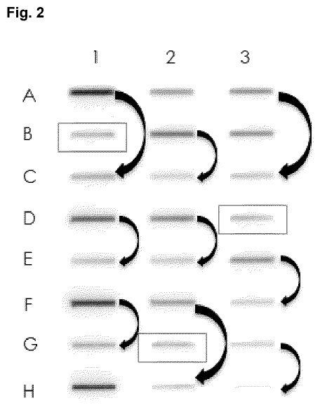

Double stranded RNA (dsRNA) byproduct formation can be decreased during in vitro transcription (IVT) by increasing Magnesium concentration in the reaction. Both for uncapped as well as CleanCap RNA, performing the IVT reaction at 24 mM MgCl₂ showed an higher amount of dsRNA formation compared with reactions performed with 55 mM Mg in de IVT (FIG. 1). A concentration of MgCl₂ to 55 mM reduced dsRNA formation on average with 62% compared to IVT reactions with 24 mM MgCl₂. dsRNA reduction was confirmed with the immunoblot utilizing the anti dsRNA J2 antibody (FIG. 2). Details of the used compositions in and quantified data corresponding to FIG. 2 can be found in the below tables:

| | 1 | 2 | 3 |
|---|---|---|---|
| A | huCD70-24 | E7-55 | TR4-24 (CC) |
| B | Ctr | E6-24 | N/A |
| C | huCD70-55 | E6-55 | TRL4-55 (CC) |
| D | huCD40L-24 | CD70-24 (CC) | Ctr |
| E | huCD40L-55 | CD70-55 (CC) | E7-24 (CC) |
| F | TLR4-24 | CD40L-24 (CC) | E7-55 (CC) |
| G | TLR4-55 | Ctr | E6-24 (CC) |
| H | E7-24 | CD40L-55 (CC) | E6-55 (CC) |
| A | 74015 | 29905 | 31888 |
| B | 18792 | 46112 | N/A |
| C | 25489 | 13479 | 17228 |
| D | 53154 | 39772 | 18061 |
| E | 19002 | 15740 | 36486 |
| F | 76535 | 34078 | 13644 |
| G | 23205 | 17787 | 11799 |
| H | 58633 | 12528 | 3820 |

### RNA Yield and Integrity

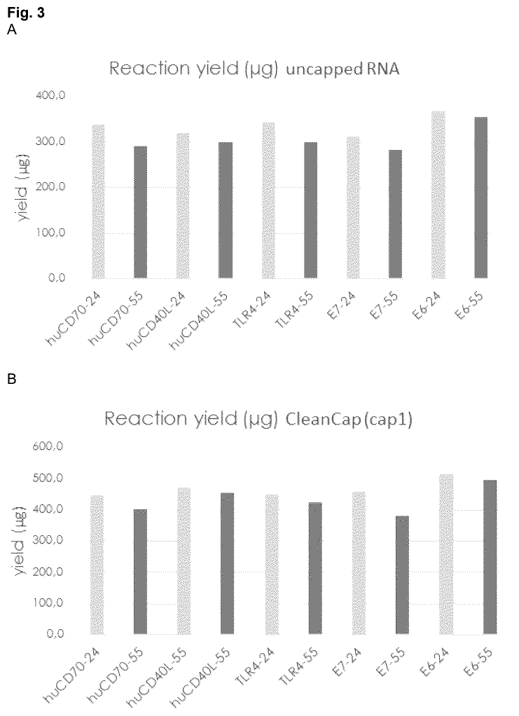

The yield of RNA after the IVT reaction is not compromised significantly in the presence of 55 mM MgCl₂ compared to reactions with 24 mM MgCl₂, both for uncapped as well as CleanCap RNA (FIG. 3. Panel A and B). In addition, the integrity of the RNA is not compromised when IVT is performed with 55 mM MgCl₂ compared to reactions with 24 mM MgCl₂ (data not shown).

### Titration Results

A further titration experiment was performed in which increasing concentrations of Magnesium were used. As evident from the below table, the dsRNA concentration decreases significantly when using increasing concentrations of Magnesium, wherein 35 mM magnesium reduces the amount of dsRNA over 50% compared to 24 mM magnesium.

| | | | Corrected |
|---|---|---|---|
| | | Calculated dsRNA | Calculated dsRNA |
| | Calculated dsRNA | content | content |
| Sample LOT | content (ng/mL) | (ng dsRNA/μg mRNA) | (ng dsRNA/μg mRNA) |
| LB7_Mg 35 | 11.81 | 2.36 | 4.72 |
| LB8_Mg 45 | 8.34 | 1.67 | 3.34 |
| LB9_Mg 55 | 6.98 | 1.40 | 2.79 |
| LB19_HiT7 Mg24 | 107.26 | 21.45 | 42.91 |
| LB20_HiT7 Mg35 | 51.32 | 10.26 | 20.53 |
| LB21_HiT7 Mg45 | 22.41 | 4.48 | 8.96 |
| LB22_HiT7 Mg55 | 18.40 | 3.89 | 7.36 |

### CONCLUSION

The addition of an elevated concentration of magnesium to the in vitro transcription mixture significantly reduced the formation of double stranded RNA. For example, a concentration of MgCl₂ to 55 mM reduced dsRNA formation on average with 62% compared to IVT reactions with 24 mM MgCl₂. The described method is compatible with the manufacturing process for different kind of mRNAs including uncapped RNA, CleanCap RNA, N1U modified RNA, RNA with and without a polyA tail. The major advantage is that additional steps to the mRNA synthesis workflow to further purify the composition can be minimized.

### REFERENCES

Mu X, Greenwald E, Ahmad S, Hur S. An origin of the immunogenicity of in vitro transcribed RNA. Nucleic Acids Res. 2018 Jun. 1; 46 (10): 5239-5249.

1-9. (canceled)

10. A method for reducing double stranded RNA (dsRNA) formation during in vitro transcription reaction, the method comprising:
preparing an in vitro transcription reaction mixture comprising:
a DNA template;
an RNA polymerase; and
a buffer system comprising at least about 35 mM magnesium; and
performing the in vitro transcription reaction to produce RNA.

11. The method according to claim 10, wherein the buffer system further comprises a pyrophosphatase.

12. The method according to claim 10, wherein the in vitro transcription reaction is terminated by the addition of a metal chelator.

13. The method according to claim 12, wherein the metal chelator is EDTA.

14. The method according to claim 10, wherein the concentration of magnesium is from about 35 mM to about 150 mM.

15. The method according to claim 10, wherein the concentration of magnesium is from about 40 mM to about 100 mM.

16. The method according to claim 10, wherein the concentration of magnesium is from about 45 mM and about 75 mM.

17. The method according to claim 10, wherein the concentration of magnesium is about 55 mM.

18. The method according to claim 11, wherein the concentration of the pyrophosphatase is from about 0.01 U/ml to about 40 U/ml.

19. The method according to claim 11, wherein the concentration of the pyrophosphatase is from about 0.1 U/ml and about 20 U/ml.

20. The method according to claim 11, wherein the concentration of the pyrophosphatase is from about 1 U/ml to about 10 U/ml.

21. The method according to claim 11, wherein the concentration of the pyrophosphatase is about 5 U/ml.

22. The method according to claim 12, wherein the concentration of the metal chelator is from about 10 mM to about 50 mM.

23. The method according to claim 12, wherein the concentration of the metal chelator is from about 20 mM to about 30 mM.

24. The method according to claim 12, wherein the concentration of the metal chelator is about 24 mM.

25. The method according to claim 10, wherein the magnesium is in a form selected from magnesium chloride or magnesium acetate.

26. The method according to claim 10, wherein the RNA comprises one or more of a 5′ CAP, one or more modified nucleosides, or a poly (A) tail.
